(19) United States
(12) Patent Application Publication
(10) Pub. No.: US 2019/0217029 A1
(43) Pub. Date: Jul. 18, 2019

(54) Dual Passageway Breathing Device

(71) Applicant: Taylor J. Lawrence, Golden, CO (US)

(72) Inventor: Taylor J. Lawrence, Golden, CO (US)

(21) Appl. No.: 16/249,617

(22) Filed: Jan. 16, 2019

Related U.S. Application Data
(60) Provisional application No. 62/617,816, filed on Jan. 16, 2018.

(51) Int. Cl. A61M 16/00; A61M 16/06; G09B 23/28; A61M 16/08

(52) U.S. Cl. CPC A61M 16/0048; A61M 16/0816; G09B 23/288; A61M 16/06

(57) ABSTRACT

A dual passageway breathing device is described. Embodiments of the dual passageway breathing device can be implemented with a cardiopulmonary resuscitation (CPR) mask. The dual passageway breathing device can allow a simulated rescuer and a simulated victim to use the CPR mask. For instance, the dual passageway breathing device can allow the simulated rescuer to practice exhaling into the mask without the simulated victim having to interface with the breaths. The dual passageway breathing device can also allow the simulated victim to breathe via the device while wearing the mask.

### CROSS-REFERENCE TO RELATED APPLICATION

This application claims the benefit of U.S. Provisional Application No. 62/617,816, filed Jan. 16^{th}, 2018.

### BACKGROUND

Currently, when training and practicing in water rescue breathing with a CPR mask, the rescuing lifeguard will perform the rescue and then place the CPR mask on the “drowning” lifeguard. The rescuer can then touch their chin to the valve of the CPR mask to mimic giving a breath or just says out loud “breath” every time they would be giving a breath if it were a real scenario.

In a currently available CPR mask, a direction of flow is towards the person wearing the mask. There is a valve (or membrane) halfway up the tube of the one-way valve that controls the flow. Any air above that membrane can only flow downward toward the mask. If the person wearing the mask breathes in, they are able to take a breath because air is flowing in the direction that the valve allows. When the person wearing the valve exhales, the membrane is pushed up from the pressure against the tube and the air escapes from vents just below the valve. If you are located downstream of the valve, you are able to both inhale and exhale, however if you are located upstream of the valve, you can only exhale. As can be appreciated, if a live person was wearing the mask while the rescuing lifeguard was breathing into the mask, the person wearing the mask would not be able to easily breathe.

A device that can allow a CPR trainee to practice with a CPR mask on a live person is needed.

### BRIEF DESCRIPTION OF THE DRAWINGS

FIGS. 1A-1D include several different views of a dual passageway breathing device according to one embodiment of the present invention.

FIG. 2 is a perspective view of a dual passageway breathing device coupled to a CPR mask according to one embodiment of the present invention.

FIGS. 3A-3L include several different views of a dual passageway breathing device according to one embodiment of the present invention.

FIG. 4A is a perspective view of a dual passageway breathing device coupled to a CPR mask according to one embodiment of the present invention.

FIG. 4B is a perspective view of a dual passageway breathing device coupled to another CPR mask according to one embodiment of the present invention.

### DETAILED DESCRIPTION

Embodiments of the present invention include a dual passageway breathing device that allows a user to practice cardiopulmonary resuscitation (CPR) on a live person with a CPR mask. Of note, most CPR masks include a one-way valve. The dual passageway breathing device can allow a user to practice giving breaths to another person while allowing said person to continue breathing normally while wearing a CPR mask.

Generally, the dual passageway breathing device can include a housing having a first passageway, a second passageway, and a barrier. An opening can be included on either end of the housing as an intake for each of the passageways. The passageways can end in an opening on either side of the housing proximate a middle of the housing. The dual passageway breathing device can be sized to be implemented with currently available CPR masks implementing valves. In one embodiment, the dual passageway breathing device can include an opening sized to restrict airflow to help simulate giving breaths while performing CPR.

In a first embodiment, a dual passageway breathing device can be implemented to replace a one-way valve of a CPR mask and couple to an intake tube of the CPR mask. The first embodiment dual passageway breathing device can allow a simulate rescuer and a simulated victim to breathe via passageways of the device.

In a second embodiment, a dual passageway breathing device can be implemented to couple to a CPR mask having a one-way valve and work in conjunction with the one-way valve and CPR mask. The second embodiment dual passageway breathing device can allow simulated victim to breathe in via the device and exhale normally through the CPR mask. Similar to the first embodiment, a simulated rescuer may practice giving breaths to the simulated victim.

In a typical CPR mask, air is administered to a patient (or victim) when a user exhales into a one-way valve that allows the air to pass to the patient. Of note, most modern CPR masks have either a built-in one-way valve or an attachable, disposable one-way valve to protect the user from the patient's potentially infectious bodily substances. As can be appreciated, a one-way valve may be implemented when practicing with a dummy, but would not be practical when practicing with a live person. More specifically, the live person would need a way to breathe as the other person is blowing air into their mouth and nose via the CPR mask. The second embodiment dual passageway breathing device can be implemented to allow two live persons to practice with a CPR mask having a one-way valve.

In one embodiment, the dual passageway breathing device can include a housing having a first portion and a second portion. The housing can include, but is not limited to, a first passageway, a second passageway, and a barrier. The first passageway can be defined by a first opening and a second opening. Typically, the first passageway can be located substantially within the first portion of the housing. The second passageway can be defined by a third opening and a fourth opening. The second passageway can be located substantially within the second portion of the housing. The barrier can be implemented to separate the first passageway from the second passageway. The first portion of the housing can be adapted to interface with a user and the second portion of the housing can be adapted to interface with a cardiopulmonary resuscitation (CPR) mask.

Embodiments of the dual passageway breathing device can allow a simulated rescuer to practice blowing into a CPR mask to give rescue breaths without breathing into a person who is pretending to drown in a training scenario. With the first embodiment dual passageway breathing device attached, the one-way valve can be replaced by the device and each person has their own airway through which they are able to breathe. With the second embodiment dual passageway breathing device, the device can attach to a top of the one-way valve of the CPR mask. Of note, this may allow the person wearing the CPR mask to breathe through the mask the same way they would without the device attached. Since the device has two separate passageways (or airways), the device can still allow the rescuer to practice blowing into the device without breathing into the person wearing the CPR mask.

Embodiments of the dual passageway breathing device can allow a simulated rescuer to practice giving breaths in a more realistic manner allowing the simulated rescuer to actually blow into the device to practice the actual technique that would be done in an emergency. Practicing this way may help develop muscle memory in rescuers (e.g., lifeguards, first responders, etc.) to improve their training so in an actual emergency when CPR skills may be needed, the rescuer will not hesitate.

In operation, the first embodiment dual passageway breathing device can be inserted into a one-way valve of a CPR mask. Alternatively, the second embodiment dual passageway breathing device can receive the one-way valve of the CPR mask. After the device has been coupled to the one-way valve, the CPR mask can be placed on a person acting as a victim. Once the CPR mask is in place, a person practicing using the CPR mask can begin using the dual passageway breathing device in combination with the CPR mask. Of note, as the person practicing giving breaths breathes into the combination, air from the person is directed out the side of the housing such that the person wearing the mask does not receive the breaths from the person practicing. The person wearing the mask can draw in breaths via the dual passageway breathing device and exhale via exhaust vents on the mask, thus being able to breathe while the other person practices giving breaths.

### Terminology

The terms and phrases as indicated in quotation marks (“ ”) in this section are intended to have the meaning ascribed to them in this Terminology section applied to them throughout this document, including in the claims, unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive; rather the term is inclusive, meaning either or both.

References in the specification to “one embodiment”, “an embodiment”, “another embodiment, “a preferred embodiment”, “an alternative embodiment”, “one variation”, “a variation” and similar phrases mean that a particular feature, structure, or characteristic described in connection with the embodiment or variation, is included in at least an embodiment or variation of the invention. The phrase “in one embodiment”, “in one variation” or similar phrases, as used in various places in the specification, are not necessarily meant to refer to the same embodiment or the same variation.

The term “couple” or “coupled” as used in this specification and appended claims refers to an indirect or direct physical connection between the identified elements, components, or objects. Often the manner of the coupling will be related specifically to the manner in which the two coupled elements interact.

The term “directly coupled” or “coupled directly,” as used in this specification and appended claims, refers to a physical connection between identified elements, components, or objects, in which no other element, component, or object resides between those identified as being directly coupled.

The term “approximately,” as used in this specification and appended claims, refers to plus or minus 10% of the value given.

The term “about,” as used in this specification and appended claims, refers to plus or minus 20% of the value given.

The terms “generally” and “substantially,” as used in this specification and appended claims, mean mostly, or for the most part.

Directional and/or relationary terms such as, but not limited to, left, right, nadir, apex, top, bottom, vertical, horizontal, back, front and lateral are relative to each other and are dependent on the specific orientation of an applicable element or article, and are used accordingly to aid in the description of the various embodiments and are not necessarily intended to be construed as limiting.

### A First Embodiment of a Dual Passageway Breathing Device

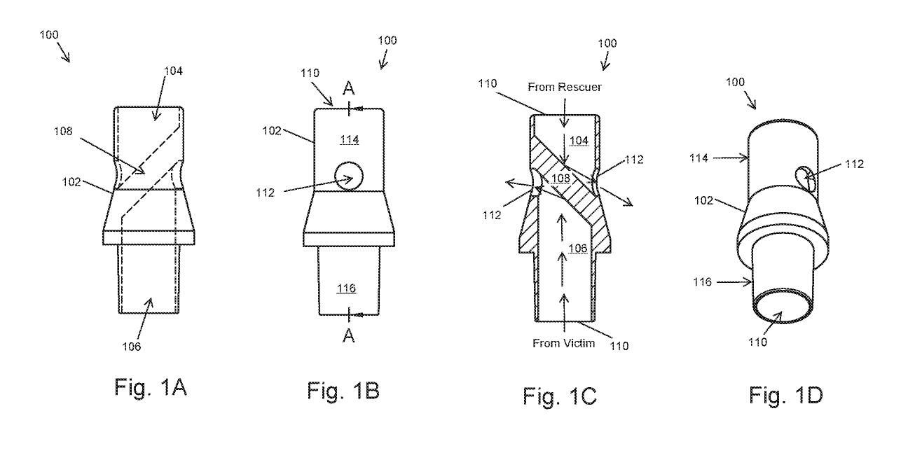

Referring to FIGS. 1A-1D, a plurality of different views of a first embodiment 100 of a dual passageway breathing device is illustrated. The first embodiment dual passageway breathing device 100 can typically be implemented with a CPR mask that does not include a one-way valve. The dual passageway breathing device 100 can be implemented to allow a user to practice giving breaths to a live person wearing a CPR mask.

Referring to FIG. 1A, a front view of the dual passageway breathing device 100 is illustrated. Referring to FIG. 1B, a side view of the dual passageway breathing device 100 is illustrated including a cross-sectional line A-A. Referring to FIG. 1C, a cross-sectional view of the dual passageway breathing device 100 along the line A-A is illustrated. Referring to FIG. 1D, a bottom perspective view of the dual passageway breathing device 100 is illustrated.

As shown generally in FIGS. 1A-1D, the dual passageway breathing device 100 can include, but is not limited to, a housing (or body) 102, a first passageway 104, a second passageway 106, and a barrier 108. Typically, each of the passageways 104, 106 can include an intake opening 110 and an exhaust opening 112. The passageways 104, 106 are shown via dotted lines in FIG. 1A and in FIG. 1C.

Generally, the housing 102 can be sized and shaped to interact with a commonly available CPR mask. As shown, the housing 102 can include a first portion 114 and a second portion 116. The first portion 114 can be configured to be used by a rescuer and the second portion 116 can be configured to be inserted into a CPR mask. Of note, the second passageway 106 can be fluidly connected to the CPR mask such that a person whom has the mask on their face can breathe via the second passageway 106.

As shown in FIGS. 1A (via dotted lines) and 1C, the housing 102 can include the interior barrier 108 between the first passageway 104 and the second passageway 106. The barrier 108 can be implemented to direct air blown into the passageways 104, 106 towards the exhaust openings 112. Of note, by having the barrier 108 at an angle, the air can be directed towards the exhaust opening 112 without directing air back towards the intake opening 110. For instance, the air can be deflected off of the barrier 108 towards the exhaust opening 112. Generally, the barrier 108 can be angled between approximately 25-65 degrees from parallel with a minor axis of the housing 102.

In some embodiments, the barrier 108 can be mechanically actuated to move from a closed position to an open position to form a single passageway directly from the intake opening of the first passageway to the intake opening of the second passageway. In such an embodiment, each of the exhaust openings can include a valve that closes when the barrier is opened to create the single passageway. Of note, in such an embodiment, the dual passageway breathing device can be implemented to practice with either a live person or a dummy.

As shown generally in FIGS. 1B-1C, the exhaust openings 112 of the passageways 104, 106 can be located on opposite sides of the housing 102. In one embodiment, the exhaust openings 112 can be located approximate an upper third of the housing 102. Of note, the exhaust openings 112 can be located anywhere along a length of the housing 102 such that the opening is not effectively or likely covered by either a mask or a mouth of a rescuer. The intake openings 110 can be fluidly connected to the respective exhaust openings 112.

As shown in FIG. 1C, a typical airflow of a breath from the mouth of a rescuer through the first passageway 104 and an airflow of a breath from the victim through the second passageway 106 is illustrated. Of note, each of the individuals interacting with the dual passageway breathing device 100 can exhale through the device 100, and if needed, each individual can breathe air in through the device. As can be appreciated, a person simulating being a rescuer can practice giving breaths to a live person while the simulated victim can breathe normal while interacting with the a CPR mask and the device 100.

As shown in FIG. 1C, the device can include two exhaust openings 112 for exhausting air, and if needed, the openings can each be reversed to act as intakes for sucking air in and exhausting air to the individuals if needed.

Generally, the second portion 116 of the housing 102 can have a smaller perimeter than the first portion 114. Where the second portion 116 meets the first portion 114 of the housing 102, a lip or protrusion extending from a perimeter of the second portion 116 can be implemented to keep the device 100 from inserting too far in a tube of a CPR mask.

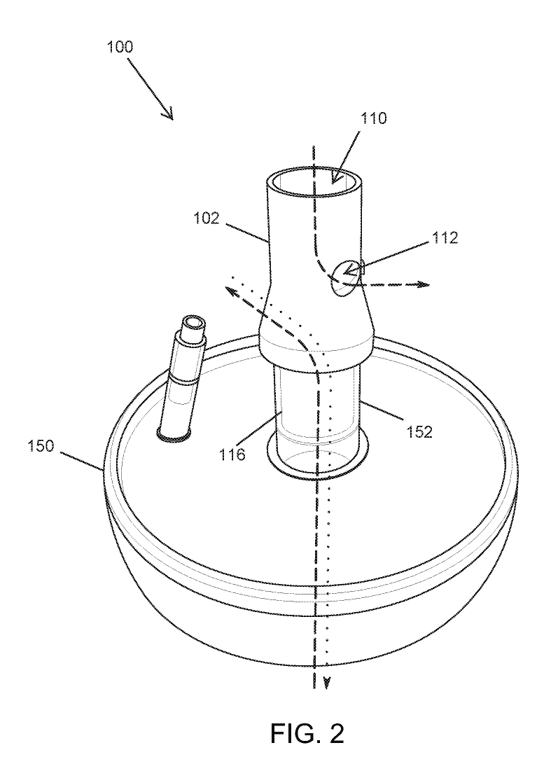

Referring to FIG. 2, a detailed diagram of the dual passageway breathing device 100 coupled to a CPR mask 150 is illustrated. As shown, the second portion 116 of the housing 102 can be inserted into a tube 152 of the CPR mask 150. Of note, the CPR mask 150 does not include a one-way valve. As previously mentioned, the first embodiment dual passageway breathing device 100 can typically be implemented with a CPR mask not including a one-way valve. As can be appreciated, a length of the housing 102 can be sized to simulate a typical length of a disposable one-way valve to allow for dimensions of the CPR mask to mimic a real-life situation.

Further shown in FIG. 2 is an airflow path through the passageways. Of note, when the CPR mask 150 does not include a one-way valve, a user wearing the mask 150 can inhale and exhale via the second passageway 106 (not shown). A user practicing giving CPR to a victim may exhale breaths via the first passageway 104 (not shown).

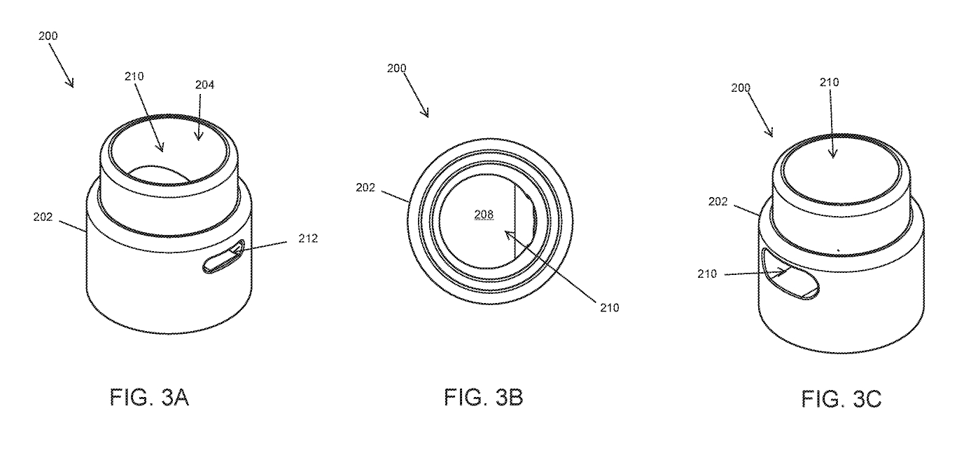

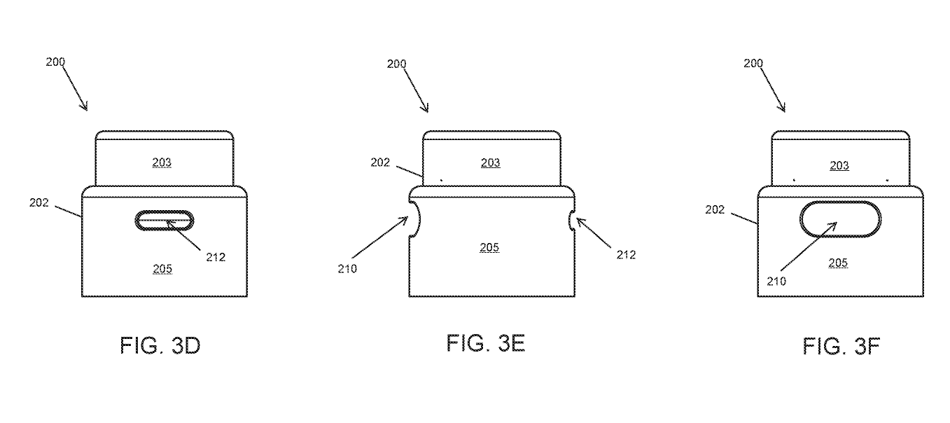

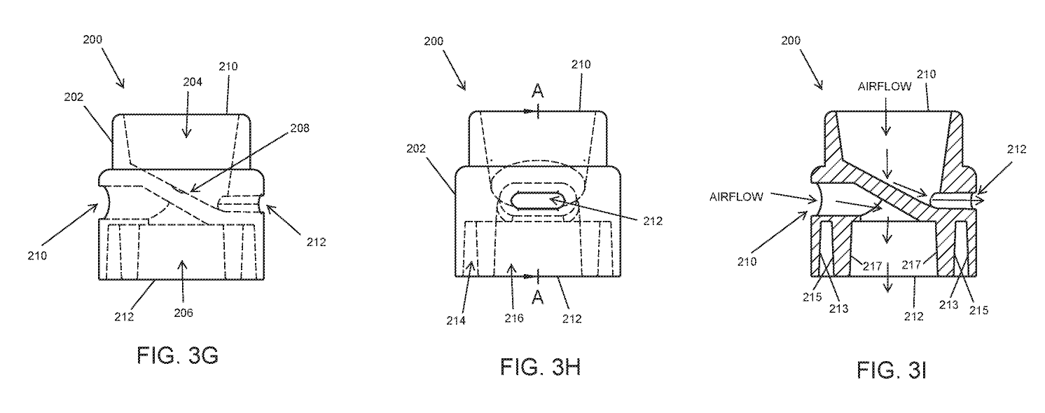

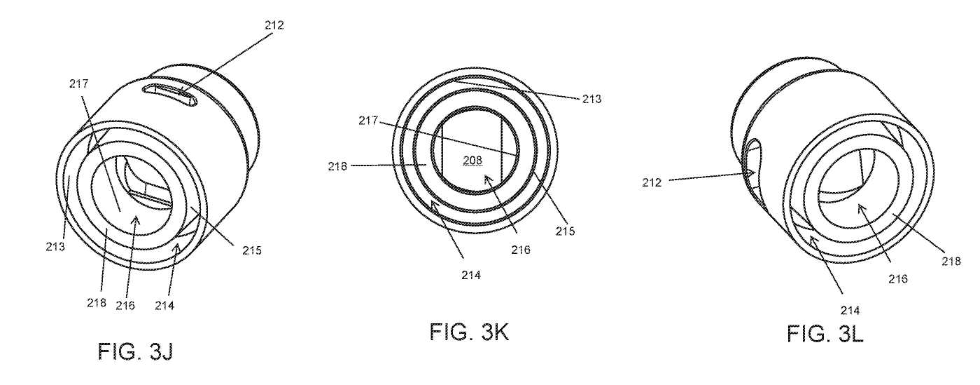

Referring generally to FIGS. 3A-3L, a second embodiment 200 of a dual passageway breathing device is illustrated. Typically, the second embodiment device 200 can be implemented with a CPR mask that has a one-way valve. In some instances, the one-way valve can be integrally formed with the mask. In other instances, the one-way valve may be removably coupled to the CPR mask. The second embodiment dual passageway breathing device 200 can be functionally similar to the first embodiment dual passageway breathing device 100, but typically have a shorter overall length. Further, the second embodiment dual passageway breathing device 200 can include an exhaust opening configured to restrict airflow to better simulate giving breaths to a live person.

As shown generally in FIGS. 3A-3L, the second embodiment dual passageway breathing device 200 can include, but is not limited to, a housing (or body) 202, a first passageway 204, a second passageway 206, and a barrier 208. Typically, the passageways 204, 206 can each include an intake opening 210 and an exhaust opening 212. Of note, the intake opening 210 and the exhaust opening 212 of the second passageway 206 can be opposite of the openings 210, 212 of the first passageway 204, as generally shown by an airflow of the passageways 204, 206 in FIG. 3I.

Typically, the housing 202 of the dual passageway breathing device 200 can be shorter in length than the first embodiment dual passageway breathing device 100. As noted in FIGS. 3D-3F, the housing 202 can include a first portion 203 and a second portion 205. Typically, the first portion 203 can include an upper third of the housing 202 and the second portion 205 can include a lower two-thirds of the housing 202.

Referring to FIG. 3A, a front perspective view of the dual passageway breathing device 200 is illustrated. The intake opening 210 and the exhaust opening 212 of the first passageway 204 is shown. The exhaust opening 212 of the first passageway 204 can be located on a side of the housing 202 approximate a middle of the housing 202. The exhaust opening 212 of the first passageway 204 can be sized to restrict airflow from a user interfacing with the first portion 203 of the housing 202. As can be appreciated, by restricting airflow, the device 200 can be implemented to simulate giving breaths to a live person when practicing CPR.

Referring to FIG. 3B, a top view of the dual passageway breathing device 200 is illustrated.

Referring to FIG. 3C, a front perspective view of the dual passageway breathing device 200 is illustrated. The intake openings 210 of the first passageway 204 and the second passageway 206 are shown. Of note, the intake opening 210 of the second passageway 206 is located on a side of the housing 202 approximate a middle of the housing 202. Typically, the intake opening 210 of the second passageway 206 can be located on an opposite side of the housing 202 from the exhaust opening 212 of the first passageway 204.

Referring to FIG. 3D, a side view of the dual passageway breathing device 200 is illustrated. As shown, the exhaust opening 212 of the first passageway 204 can be sized to restrict an airflow received via the intake opening 210 of the first passageway 204. As can be appreciated, a size of the exhaust opening 212 may be changed to increase or decrease an airflow through the first passageway 204.

Referring to FIG. 3E, a front view of the dual passageway breathing device 200 is illustrated. As shown, the intake opening 210 of the second passageway 206 can be on an opposite side of the housing 202 from the exhaust opening 212 of the first passageway 204. Typically, the intake opening 210 of the second passageway 204 and the exhaust opening 212 of the first passageway 204 can be located approximate a middle exterior of the housing 202.

Referring to FIG. 3f, a side view of the dual passageway breathing device 200 is illustrated. Typically, the intake opening 210 of the second passageway 204 can allow for maximum airflow to allow a wearer of a CPR mask to breathe easily while simulating being a victim.

Referring to FIG. 3G, a front view of the second embodiment dual passageway breathing device 200 is illustrated. Typically, the intake opening 210 for the first passageway 204 can be located proximate a top of the housing 202 and the intake opening 210 for the second passageway 206 can be located proximate a side of the housing 202. As shown by dotted lines, the exhaust opening 212 for the first passageway 204 can be located on a side of the housing and the exhaust opening 212 for the second passageway 206 can be located proximate a bottom of the housing 202. Of note, by exhausting the air to the side of the housing 202, air blown through the first passageway 204 can be directed away from the users of the dual passageway breathing device 200.

The housing 202 can include the barrier 208 to separate the two passageways 204, 206. In one embodiment, the barrier 208 can be angled at approximately 45 degrees from parallel with a minor axis of the housing 202. By having the barrier 208 angled, air blown into the passageways 204, 206 can be efficiently deflected and directed out to the exhaust openings 212. Generally, the barrier 208 can be angled between approximately 25-65 degrees from parallel with a minor axis of the housing 202. As can be appreciated, an angle of the barrier 208 can be altered without exceeding a scope of the present invention.

Referring to FIG. 3H, a side view of the dual passageway breathing device 200 is illustrated. FIG. 3H includes a cross-sectional line A-A. As previously mentioned, the exhaust opening 212 of the first passageway 204 can be sized to restrict an airflow through the first passageway 204.

Referring to FIG. 3I, a cross-sectional view along line A-A from FIG. 3H is illustrated. FIG. 3I further includes an example flow path of air through the first passageway 204 and the second passageway 206. As shown, air entering the intake opening 210 of the first passageway 204 can be deflected off of the barrier 208 towards the exhaust opening 212 to vent the air to atmosphere. Of significant note, the second passageway 206 can have an airflow in an opposite direction of the airflow of the first passageway 204. As shown, air entering the intake opening 210 of the second passageway 206 can be deflected off of the barrier 208 towards a bottom of the housing 202 and the exhaust opening 212 of the second passageway 206. As previously noted, the second passageway 206 can allow the simulated victim to breathe air in when a CPR mask includes a one-way valve. Typically, a one-way valve of a CPR mask allows air from a rescuer to pass through the valve to the victim and does not allow air to pass from the victim to the rescuer.

Referring generally to FIGS. 3G-3L, a means for the dual passageway breathing device 200 to couple to different sized tubes is shown. The dual passageway breathing device 200 can be sized to receive a tube of an existing one-way valve attached to a CPR mask. As shown in dotted lines in FIGS. 3G-3H, the cross-sectional view of FIG. 3I, and in FIGS. 3J-3L, the housing 202 can be configured to receive tubes having different sizes. The exhaust opening 212 of the second passageway 206 can include a first receptacle 214 and a second receptacle 216. The first receptacle 214 can be defined by an interior wall 213 of the housing 202 and an exterior wall 215 of an interior protrusion 218 of the housing 202. The second receptacle 216 can be defined by an interior wall 217 of the interior protrusion 218. The first receptacle 214 can have a larger perimeter than the second receptacle 216. As can be appreciate, the first receptacle 214 can be configured to receive a larger sized tube than the second receptacle 216.

Referring to FIG. 3J, a bottom perspective view of the second embodiment dual passageway breathing device 200 is illustrated. Of note, the first receptacle 214 and the second receptacle 216 can each be sized and shaped to receive a tube from a CPR mask.

Referring to FIG. 3K, a bottom view of the second embodiment dual passageway breathing device 200 is illustrated. As shown, the second receptacle 216 can have a smaller perimeter than the first receptacle 214.

Referring to FIG. 3L, a bottom perspective view of the second embodiment dual passageway breathing device 200 is illustrated.

In a typical implementation, the second passageway 206 can be used by a person simulating being a victim. As can be appreciated, the simulated victim can inhale air via the second passageway 206 and the one-way valve in lieu of breaths from the person. Of significant note, the exhaust opening 212 of the first passageway 204 can be smaller than the exhaust opening 212 of the second passageway 206. The smaller exhaust opening can be implemented to restrict airflow from the rescuer blowing into the device 200 to simulate a pressure needed to breathe into someone when giving CPR or rescue breathing procedures.

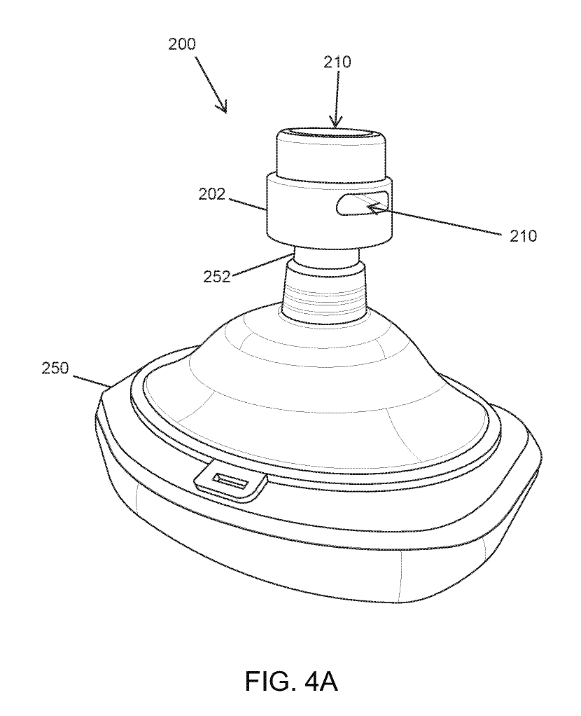

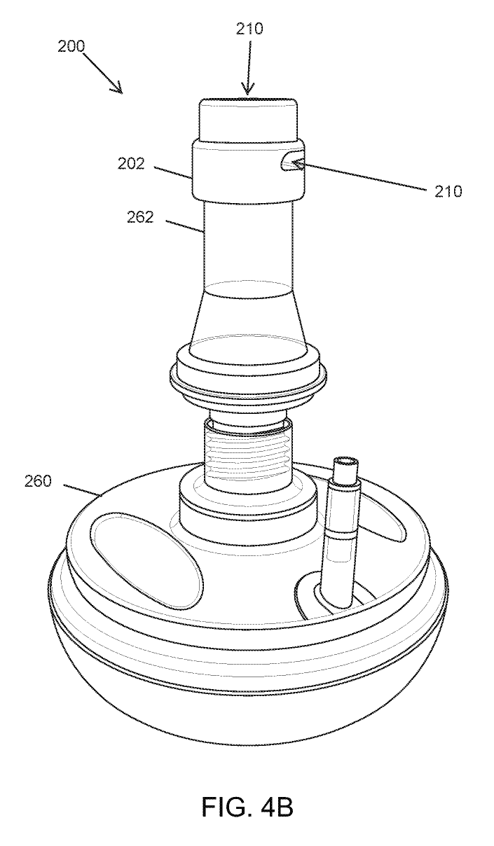

Referring to FIGS. 4A-4B, detailed diagrams of the second embodiment dual passageway breathing device 200 coupled to different CPR masks are illustrated. FIG. 4A shows the dual passageway breathing device 200 coupled to a CPR mask 250 that includes a tube 252 sized to fit into the second receptacle 216 of the housing 202. FIG. 4B shows the dual passageway breathing device 200 coupled to a CPR mask 260 that includes a tube 262 sized to fit into the first receptacle 214 of the housing 202.

Described hereinafter is one example method of implementing either the first embodiment dual passageway breathing device 100 or the second embodiment dual passageway breathing device 200. Hereinafter, the second embodiment device 200 will be referred to when describing the method of implementing the dual passageway breathing devices. It is to be appreciated that the devices 100, 200 can be implemented in a substantially similar manner.

In a first step, the dual passageway breathing device 200 can receive a tube of a CPR mask.

In a second step, a simulated rescuer can place the CPR mask with the device 200 attached proximate a face of a simulated victim. For instance, a person may simulate having drowned in a pool and the simulated rescuer must perform CPR to save their life.

In a third step, the simulated rescuer can interface directly with the device 200 and practice giving breaths in a CPR exercise. As the simulated rescuer breathes into the device 200, the breaths can pass through the first passageway 204 and vent to atmosphere via the exhaust opening 212 of the first passageway 204. As such, the simulated victim wearing the mask does not interface with the simulated rescuers breaths. The simulated victim can inhale air via the intake opening 210 of the second passageway 206. If a one-way valve is included with the CPR mask, the simulated victim may exhale via vents of the CPR mask. If the CPR mask does not include a one-way valve, the simulated victim can inhale and exhale via the openings 210, 212 of the second passageway 206.

### Alternative Embodiments and Variations

The various embodiments and variations thereof, illustrated in the accompanying Figures and/or described above, are merely exemplary and are not meant to limit the scope of the invention. It is to be appreciated that numerous other variations of the invention have been contemplated, as would be obvious to one of ordinary skill in the art, given the benefit of this disclosure. All variations of the invention that read upon appended claims are intended and contemplated to be within the scope of the invention.

I claim:

1. A dual passageway breathing device comprising:
a housing having a first portion and a second portion, the housing including:
a first passageway including a first opening and a second opening, the first passageway being located substantially within the first portion of the housing;
a second passageway including a third opening and a fourth opening, the second passageway being located substantially within the second portion of the housing; and
a barrier separating the first passageway from the second passageway;
wherein the first portion of the housing is adapted to interface with a user and the second portion of the housing is adapted to interface with a cardiopulmonary resuscitation mask.

2. The dual passageway breathing device of claim 1, wherein (i) the first opening is located approximate a top of the housing, and (ii) the second opening is located approximate a middle exterior of the housing.

3. The dual passageway breathing device of claim 1, wherein (i) the third opening is located approximate a bottom of the housing, and (ii) the fourth opening is located approximate another middle exterior of the housing.

4. The dual passageway breathing device of claim 1, wherein a fluid entering the first opening passes through the first passageway to exit the second opening and vice versa.

5. The dual passageway breathing device of claim 1, wherein a fluid entering the third opening passes through the second passageway to exit the fourth opening and vice versa.

6. The dual passageway breathing device of claim 1, wherein the second portion of the housing includes (i) a first receptacle being defined by an interior wall of an interior protrusion, and (ii) a second receptacle being defined by an exterior wall of the interior protrusion and an interior wall of the housing.

7. The dual passageway breathing device of claim 6, wherein a perimeter of the first receptacle is smaller than a perimeter of the second receptacle.

8. The dual passageway breathing device of claim 1, wherein the second opening and the fourth opening are each located on a sidewall of the housing.

9. The dual passageway breathing device of claim 1, wherein the barrier is oriented at approximately 45 degrees from parallel with a minor axis of the housing.

10. A dual passageway breathing device for use with a cardiopulmonary resuscitation (CPR) mask, the device comprising:
a first portion adapted to interface with a user;
a second portion adapted to couple to a CPR mask;
a first opening fluidly connected to a second opening forming a first passageway, the first passageway being located substantially within the first portion;
a third opening fluidly connected to a fourth opening forming a second passageway, the second passageway being located substantially within the second portion;
a barrier located internally of the device, the barrier separating the first passageway from the second passageway.

11. The dual passageway breathing device of claim 10, wherein the device is coupled to a CPR mask.

12. The dual passageway breathing device of claim 10, wherein (i) the first opening is located proximate a top of the device, and (ii) the second opening is located proximate a middle side of the device.

13. The dual passageway breathing device of claim 10, wherein (i) the third opening is located proximate a bottom of the device, and (ii) the fourth opening is located proximate a middle side of the device.

14. The dual passageway breathing device of claim 10, wherein the barrier is oriented between 25-65 degrees from parallel with a minor axis of the device.

15. The dual passageway breathing device of claim 10, wherein a user interfacing with the first portion of the device exhales via the first passageway.

16. The dual passageway breathing device of claim 15, wherein a user wearing the CPR mask inhales and exhales via the second passageway.

17. The dual passageway breathing device of claim 10, wherein (i) the first opening is bigger than the second opening, and (ii) the third opening is bigger than the fourth opening.

18. The dual passageway breathing device of claim 10, wherein the third opening is adapted to couple to the CPR mask.

19. The dual passageway breathing device of claim 10, wherein the second opening and the fourth opening are located on opposite sides of the device.

20. A dual passageway breathing device comprising:
a first end adapted to interface with a user;
a second end adapted to interface with a CPR mask;
a first passageway having a first opening and a second opening, the first opening being located proximate a top of the device and the second opening being located proximate a middle side of the device;
a second passageway having a third opening and a fourth opening, the third opening being located proximate a bottom of the device and the fourth opening being located proximate a middle side of the device; and
a barrier separating the first passageway from the second passageway;
wherein (i) a user interfacing with the first end of the device exhales via the first passageway, and (ii) a user wearing the CPR mask inhales via the second passageway.
